(19) United States
(12) Patent Application Publication
(10) Pub. No.: US 2008/0069872 A1
(43) Pub. Date: Mar. 20, 2008

(54) Multi-Layered Controlled-Release Methylphenidate Pellet

(75) Inventors: Guillermo Rubio Badia, Castellbisbal (ES); Manuel Roig Carreras, Calafell (ES); Ramon Salazar Macian, Barcelona (ES); Pere Tubau Arino, Sant Feliu De Llobregat (ES); Nuria Rubio Rosell, Barcelona (ES); Oscar Sanchez Vega, Cornella De Llobregat (ES)

Correspondence Address: SUGHRUE MION, PLLC, 2100 PENNSYLVANIA AVENUE, N.W., SUITE 800, WASHINGTON, DC, 20037, US

(73) Assignee: LABORATORIOS RUBIO, S.A., Castellbisbal (ES)

(21) Appl. No.: 11/628,965

(22) PCT Filed: Jun. 1, 2005
(86) PCT No.: PCT/EP05/05874

§ 371 Date: Dec. 8, 2006

(30) Foreign Application Priority Data
Jun. 10, 2004 (ES) ES P 200401412

(51) Int. Cl. A61K 9/52; A61K 31/445; A61P 25/00; A61K 9/24

(52) U.S. Cl. 424/457; 424/472; 424/497; 514/317

(57) ABSTRACT

The present invention relates to a type of controlled-release multilayered methylphenidate pellet that does not need to be combined with another type of pellet. It allows the maintenance of therapeutic levels in the plasma during 12 hours with a single daily dose, avoiding repeated administrations throughout the day. The controlled-release multi-layered pellet is comprised of an inert core, a first layer that contains methylphenidate and an acid buffering system, a protective layer, a layer of ethylcellulose, that performs the function of controlling the extended release of most of the methylphenidate, and a second layer of methylphenidate, that is responsible for the immediate release of the aforesaid within one hour of administration. The weight ratio between the methylphenidate present in the first active layer and the ethylcellulose is between 1.40:1 and 1.90:1. The multi-layered pellet may possibly have an external coating to protect it from erosion during processing.

### FIELD OF THE INVENTION

The present invention relates to a type of multi-layered controlled-release methylphenidate pellet that allows administration in a single daily dose.

### PRIOR STATE OF THE ART

Methylphenidate, a piperidine derivative, with a stimulating activity on the central nervous system and breathing, which is currently marketed in the hydrochloride form to treat attention deficit disorder with hyperactivity in children.

Methylphenidate is absorbed immediately in the gastrointestinal tract, and the effects last for 3 to 6 hours. Therefore, in order to maintain therapeutic levels in the plasma, 5 to 10 mg doses administered two or three times per day are required. This is a drawback, especially in schoolchildren, due to the repetition of administration throughout the day.

Excessively high doses can cause side affects, due to the immediate release of methylphenidate.

These types of doses various times per day have been replaced by 20 mg methylphenidate 8-hour controlled-release forms, which maintain levels in the plasma with a single daily dose.

U.S. Pat. No. 6,344,215B1 discloses that they have been able to improve the effectiveness of the preparation by administering 20 mg of methylphenidate in gelatine capsule form. These capsules contain a total of 14 mg methylphenidate in two different types of pellets: an immediate release group (30%) containing 6 mg of active ingredient, and an other extended release group (70%). This method achieves both a rapid action and a prolonged effect over 12 hours, allowing administration in a single daily dose.

The preparation of gelatine capsules containing two different types of pellets complicates the production and dosage process.

Patent application WO-AI-9903471 explicitly discloses preparations with two different pellets and states the possible alternative of multi-layered pellets wherein the innermost layer, that contains the methylphenidate for extended release, is coated in a layer of ammonium methacrylate polymer and, on top of this, has another layer that contains methylphenidate for immediate release. Nevertheless, said patent application does not explicitly disclose production of multi-layered pellets.

According to that disclosed in Padmanabhan, Analytical Profiles of Drugs Substances, 10:433-497 (1981), methylphenidate is hydrolyzed in non-acidic conditions to produce practically inactive ritalinic acid. Therefore when methylphenidate is released in the intestine, at a pH of more than 5, its therapeutic activity is reduced since methylphenidate is in an environment that produces its hydrolysis.

Therefore there is a need to have a different form of controlled-release methylphenidate that allows a single daily administration with a prolonged 12 hour effect, simplified production and dosage process and which is more stable in non-acidic environments.

The authors of the present invention have discovered a type of controlled-release multi-layered methylphenidate pellet that does not need to be combined with another type of pellet and that allows the administration of a single daily dose, simplifying the production and dosage processes and which has a better stability when in non-acidic environments.

### OBJECT OF THE INVENTION

The object of this invention is a type of controlled-release multi-layered methylphenidate pellet that does not need to be combined with another type of pellet and that is more stable in non-acidic environments.

Another object of this invention is the preparation procedure for controlled-release multi-layered methylphenidate pellets.

Pharmaceutical forms that contain the aforementioned multi-layered pellets also form part of the object of this invention.

### DETAILED DESCRIPTION OF THE INVENTION

The controlled-release multi-layered methylphenidate pellet invention is characterised in that it comprises:
An inert core,
An active first layer that makes up between 65% and 75% in weight of the total methylphenidate, a filmogenic substance, a plastificizer and an acid buffering system adjusted to a pH value of between 4 and 5,
Protective layer,
Layer of plastified ethylcellulose, and
A second active layer that comprises between 25% and 35% in weight of the total methylphenidate, a filmogenic substance and a plasticizer,
   Therefore the weight ratio between the methylphenidate present in the first active layer and the ethylcellulose is between 1.40:1 and 1.90:1.

According to another characteristic of the invention, the methylphenidate controlled-release multi-layered pellet also has an external coating.

Unless otherwise specified, in this description the term methylphenidate is used in a wide sense and includes its pharmaceutical salts, such as for example methylphenidate hydrochloride.

When preparing the multi-layered pellet invention different levels of filmogenic and plasticizing substances are used. To avoid repetition throughout this description here will define the filmogenic and plasticizing substances that can be used to carry out the aim of the invention.

A filmogenic substance is a substance that is capable of forming a film and that is used to affix a new layer on an already existing substrate. The filmogenic substance can be chosen from: polyvinylpyrrolidone, polyoxyethylene, polyoxypropylene, hydroxypropyl methylcellulose, and hydroxypropylcellulose or mixtures of the above.

A plasticizer is a substance that is normally used to improve the mechanical properties of a film formed by a polymeric substance.

The plasticizer can be chosen from amongst: polyethylene glycol, polypropylene glycol, triacetin, tributyl citrate, dibutyl sebacate, medium chain length triglyceride fatty acids, resin acid, long chain fatty acids or mixtures thereof.

The plasticizer content can comprise between 3% and 30% in weight, although more typically between 10% and 25% in weight of the filmogenic substance.

Commercial products can also be used that are made of both filmogenic and plasticizing substances. An example of the aforementioned products is OPADRY CLEAR, marketed by the company COLORCON, which is made of hydroxypropyl methylcellulose, polyethylene glycol 400 and polyethylene glycol 6000. The hydroxypropyl methylcellulose acts as a filmogenic substance and the mixture of polyethylene glycols as a plasticizer.

The Inert Core

The methylphenidate controlled-release multi-layered pellet object of the invention has an inert core whereon the different layers are based.

Inert core is understood to mean a core that is chemically and pharmaceutically inert and that does not interact with methylphenidate and does not affect its stability.

The inert core can be made of any of the materials that the person skilled in the art knows, such as for example: sucrose, starch, microcrystalline cellulose, or combinations thereof. Microcrystalline cellulose is preferably used.

The inert cores used to make the invention will preferably have a diameter of between 700 and 1000 microns.

Microcrystalline cellulose is available on the market in different fractions according to the granulometry of the particles, for example, under the name CELLETS, marketed by the company PHARMATRANS SANAQ.

CELLETS 700 is an example of the above whose microcrystalline particles have a diameter of between 700 and 1000 microns, and additionally a minimum of 96% of the particles comply with this specification.

The First Active Layer

The first active layer is deposited on the inert core, comprising one part of the methylphenidate, the acid buffering system, a filmogenic substance and a plasticizer.

The methylphenidate present in the first layer comprises between 65% and 75% in weight of the total methylphenidate present in the capsule, preferably around 70% in weight.

For every 100 g of inert cores there will be a methylphenidate hydrochloride dose on this first active layer of between 17 and 21 g.

The methylphenidate hydrochloride present in this first active layer is that which will be released gradually.

The acid buffering system that is incorporated in this layer stabilises the methylphenidate in a non-acidic environment, for example, when intestinal alkaline liquid penetrates the capsule, avoiding the premature hydrolysis of the methylphenidate to ritalinic acid, a practically inactive metabolite.

The acid buffering system can be comprised of, for example, an organic acid combined with a physiologically acceptable organic base or a mixture of alkaline hydrogen phosphates. The acid buffering system should be preferably selected from the following group: citric acid and citrate combination, citric acid and glycine combination, glutaric acid and glycine combination, monosodium phosphate and disodium phosphate combination, monopotassium phosphate and dipotassium phosphate combination. The preferred mixture for the acid buffering system is that made of citric acid and glycine.

The citric acid can be used both in its anhydrous and monohydrate form.

The ratio of the acid buffering system components is designed through formulas and/or tables well known to the person skilled in the art, to achieve an acid buffering system adjusted to a pH value of between 4 and 5.

For example, in the specific case of the acid buffering system comprised of citric acid and glycine, the weight ratio between the monohydrate citric acid and the glycine is approximately 1:2.

The quantity of the buffering system incorporated into this first active layer of the multi-layered capsule is at least enough for the aqueous mixture of the active ingredient, filmogenic substance and plasticizer to have a pH value of between 4 and 5.

The acid buffering system that is comprised of citric acid, as well as stabilising the methylphenidate in non-acidic environments, can also act as a sequestrant for heavy metals, which could possibly be present in the composition.

The methylphenidate and acid buffering system layer is fixed to the inert core using a filmogenic and plasticizing substance, as stated above.

The amount of filmogenic substance present in this layer is that required to obtain a complete coating of the inert core. In general, using between 5 and 7 g of fimogenic substance for every 100 g of inert cores is sufficient.

The preferred filmogenic substance used is hydroxypropyl methylcellulose.

The filmogenic substance is combined with a plasticizer to improve the adhesiveness to the inert core and to achieve a complete and even coating of the aforesaid.

The preferred plasticizer is polyethylene glycol. The ideal plasticizer would be a mixture of polyethylene glycol 400 and polyethylene glycol 6000.

A marketed mixture of a filmogenic substance combined with plasticizers, such as OPADRY CLEAR, can be used.

The Protective Layer

This protective layer isolates the methylphenidate from the alkaline environment caused by the ethylcellulose layer that will be applied later, since ethylcellulose is normally marketed as an aqueous alkaline dispersion.

The protective layer, that does not contain methylphenidate, is placed on the first layer of methylphenidate.

The protective layer is made of a filmogenic and plasticizing substance.

The quantity of filmogenic and plasticizing substance present in this protective layer is that necessary to completely coat the first active layer. In general, between 2 and 3 g of protective layer for every 100 g of inert cores can be applied.

The preferred filmogenic substance is hydroxypropyl methylcellulose, and the plasticizer a mixture of polyethylene glycol 400 and polyethylene glycol 6000.

As a source of filmogenic and plasticizing substances a marketed product called OPADRY CLEAR, can also be used, as mentioned above.

The Plastified Ethylcellulose Layer

The plastified ethylcellulose serves to regulate the release of methylphenidate allowing the most part to be released steadily over 12 hours.

The weight ratio between the methylphenidate present in the first active layer the ethylcellulose is between 1.40:1 and 1.90:1, preferably between, 1.50:1 and 1.80:1.

The ethylcellulose layer is plastic coated so that it can form a flexible film and coat the surface of the underlying layer evenly.

The plastified ethylcellulose layer can be obtained through the application of an ethylcellulose dispersion with an added plasticizer, or a marketed dispersion of plastified ethylcellulose can be used, since it already contains the plasticizer.

In the event of using a dispersion of pre-plastified ethylcellulose, a product called SURELEASE from the company COLORCON can be used, that is comprised of an anhydrous ammonia dispersion, with an alkaline pH, of plastified ethylcellulose with medium chain length triglyceride fatty acids and oleic acid. This product can be used directly or diluted with water to deposit the ethylcellulose layer on the protective layer.

The Second Active Layer

A second methlyphenidate layer that has a lower proportion of active ingredient which is to be released immediately, i.e. within one hour of administration, is applied on top of the ethylcellulose layer that is responsible for the steady release of the methylphenidate.

In this case, it is not necessary to apply a protective layer between the ethylcellulose layer and the second layer of methylphenidate, since the drying process that the pellets are subject to after the incorporation of the new layer removes all the ammonia present in the aqueous dispersion of ethylcellulose. Therefore, once it is dry, the ethylcellulose layer does not produce an alkaline reaction.

The second layer of methylphenidate applied on top of the ethylcellulose layer comprises methylphenidate, a filmogenic substance and a plasticizer.

As stated above, the content of the methylphenidate present in this second active layer comprises between 25 and 35% in weight of the total methylphenidate present in the pellet, preferably around 30% in weight.

In this second active layer between 7 and 9 g of methylphenidate hydrochloride will be administered for every 100 g of inert cores.

A filmogenic substance is used to affix the methylphenidate to the underlying ethylcellulose layer, preferably hydroxypropyl methylcellulose, modified with plasticizers to improve the properties of the film that it forms.

Polyethylene glycol is the preferred plasticizer. More preferably, the plasticizer would be a mixture of polyethylene glycol 400 and polyethylene glycol 6000.

The marketed product OPADRY CLEAR can also be used, as already mentioned above.

In general, the content of OPADRY CLEAR in this layer can comprise between 2.5 and 3.5 g of every 100 g of inert cores.

The External Coating

The multi-layered pellet invented could possibly be given an external coating that protects it from erosions during the production and dosage process. This external coating could be made of a filmogenic substance, pigments and a plasticizer.

Titanium dioxide is the preferred pigment, and it is adhered to the second layer of methylphenidate by a filmogenic substance combined with plasticizers, whereby the use of the product OPADRY WHITE, marketed by the company COLORCON is preferred, which consists of hydroxypropyl methylcellulose, polyethylene glycol 400, polyethylene glycol 6000 and titanium dioxide.

The controlled-release multi-layered methylphenidate pellet object of the invention can form part of pharmaceutical administration methods that allow the use of pellets, such as for example hard gelatine capsules or tablets.

In the case of the capsules, the multi-layered pellets are administered so that every capsule has the established dose of methylphenidate to be able to maintain therapeutic levels of methylphenidate in the plasma.

In the specific case of methylphenidate hydrochloride, the capsules can contain between 10 mg and 40 mg of said active ingredient, preferably 20 mg.

Part of this invention is also comprised of a procedure for the preparation of multi-layered extended release methylphenidate pellets, that is comprised of the following stages:
The inert core is coated with an initial active layer through the application of an aqueous solution that makes up between 65% and 75% in weight of the total methylphenidate, a filmogenic substance, a plasticizer and an acid buffering system adjusted to a pH value of between 4 and 5,
a protective layer is applied,
a layer of plastified ethylcellulose is added, so that the ratio in weight between the methylphenidate present in the first active layer and the ethylcellulose is between 1.40:1 and 1.90:1, and
then a second active layer is added that contains between 25% and 35% in weight of the total methylphenidate, a filmogenic substance and a plasticizer.

The procedure for the preparation of extended release multi-layered methylphenidate pellets according to the invention, also includes a stage wherein the pellet formed is given an external coating.

The whole process of obtaining the multi-layered pellets is carried out on an apparatus such as the Würster Fluid Bed System.

The first layer of methylphenidate is applied on to the dry inert cores through a dose in an aqueous solution that is comprised of: methylphenidate, filmogenic and plasticizing substances, adjusted to a pH value of between 4 and 5 with the buffering system. Then, the pellets are dried to evaporate the water that has been included when measuring out the solution.

Later, an aqueous solution of filmogenic and plasticizing substance is measured out, to create a protective layer on the first layer of methylphenidate. When the solution has been measured out, the pellets are dried so that the water used is evaporated.

Afterwards, an aqueous dispersion of ethylcellulose, comprised of plasticizers, is applied to the dry pellets so that the weight ratio between the methylphenidate present in the first active layer and the ethylcellulose is between 1.40:1 and 1.90:1. The pellets are then dried so that the water and ammonia that come from the aqueous alkaline dispersion of ethylcellulose evaporates.

The dry pellets are coated with the second immediate release active layer through the application of an aqueous solution of methylphenidate, and filmogenic and plasticizing substance.

The preparation procedure of the composition of extended release methylphenidate can possibly include an outer coating stage.

The application of the external coating is carried out through dosing an aqueous dispersion of filmogenic substance, pigments, and plasticizers, followed by drying.

Once all the layers have been applied, the pellets are kept for at least two hours at a temperature of between 50° C. and 70° C. in order to aid the coalescence of the ethylcellulose layer and the consolidation of the different layers of the pellet. Thus, the occasional defects in the layers of the pellet that could prevent the required release profile to maintain the therapeutic levels in the plasma of methylphenidate, are avoided.

The pellets can be measured out in hard gelatine capsules of about 10 mg to 40 mg of methylphenidate hydrochloride per capsule, preferably 20 mg.

The pellets produced according to the invention process can be used for the preparation of medicines for attention deficit hyperactivity disorder, behavioural disorders, for the treatment of mild depression and narcolepsy.

The multi-layered pellet object of the invention that is comprised of an active layer for immediate release and another active layer for extended release, is suitable for the controlled-release of methylphenidate, maintaining the therapeutic levels of methylphenidate in the plasma with a single daily dose, without needing to be combined with other types of pellets. Similarly, the stability of the active ingredient when faced with non-acid environments is assured through the acid buffering system incorporated into the layer with the active ingredient responsible for prolonging the release of methylphenidate until 12 hours after administration.

The examples given below are shown so as to provide the expert in the field with a detailed explanation of the specific stages within the invention.

### EXAMPLES 1 TO 4

### Producing the Controlled-Release Methylghenidate Pellet

1,800 g of microcrystalline cellulose pellets (CELLETS 700) are put in a Würster type fluid bed system dryer and are heated at 60° C. for 120 minutes. After which time they are cooled to 45° C.

The first methylphenidate layer is applied to the dried pellets using the application of a buffered solution of the methylphenidate hydrochloride which has been prepared by dissolving the following components in 2,125 g of deionised water: 344 g methylphenidate hydrochloride, 131.3 g of OPADRY CLEAR, and it is adjusted to a pH of between 4 and 5 by adding 8.3 g of monohydrate citric acid and 16.9 g of glycine.

The buffered solution of methylphenidate hydrochloride is doses at 9 g/min. The air intake temperature is 65° C. and the product is maintained at a temperature of 45° C. The spray pressure is 15.9 Pa. These conditions are maintained in every one of the later applications.

Once the first layer of methylphenidate has dried, a protective layer of OPADRY CLEAR is applied using 46 g of OPADRY CLEAR dissolved in 925 g of deionised water.

After the protective layer of OPADRY CLEAR has dried, a layer of plastified ethylcellulose is applied using 1,126.4 g of an aqueous dispersion of SURELEASE, that corresponds to a weight ratio between the methylphenidate present in the first active layer and ethylcellulose of 1.63:1.

Once the ethylcellulose layer is dried, a second layer of methylphenidate is applied through adding a pre-prepared solution of 147.4 g methylphenidate hydrochloride and 56.3 g OPADRY CLEAR dissolved in 1,105.8 g deionised water.

After applying all the layers, the pellets are dried at a temperature of 60° C. for 2 hours.

The pellets produced are measured out at 115 mg for every hard gelatine capsule, to give a dose of 20 mg methylphenidate hydrochloride. If other doses of methylphenidate hydrochloride are desired, the corresponding doses of pellets per capsule will be measured out.

Following the same procedure described in this example, but using different weight ratios of the methylphenidate present in the first active layer and ethylcellulose, controlled methylphenidate release in examples 2 to 4 have been prepared, shown in Table 1:

TABLE 1

| Example | Weight Ratio |
|---|---|
| 2 | 1.65 |
| 3 | 1.52 |
| 4 | 1.41 |

The methylphenidate controlled-release profile of the multi-layered pellets is determined following the instructions from the trials for solution for solid forms of doses described on page 194 of the European Pharmacopoeia, Fourth Edition (2001).

Methylphenidate hydrochloride is determined through the analytical technique of high performance liquid chromatography (HPLC) through experimental conditions that can be routinely established by someone skilled in the art.

The release profile for methylphenidate corresponds to those in Examples 1 to 4, expressed as a % in weight of methylphenidate released at a specific time, as shown in Table 2:

TABLE 2

| Time (hours) | Example 1 | Example 2 | Example 3 | Example 4 |
|---|---|---|---|---|
| 0 | 0 | 0 | 0 | 0 |
| 1 | 33.8 | 35.83 | 34.50 | 30.95 |
| 2 | 40.8 | 48.54 | 37.93 | 34.64 |
| 4 | 63.7 | 70.07 | 50.52 | 39.32 |
| 8 | 91.9 | 90.87 | 79.63 | 64.84 |
| 12 | 104.1 | 99.01 | 95.72 | 83.24 |

You can see that in the four examples corresponding to the invention, you achieve a controlled-release of methylphenidate that lasts for at least 12 hours.

The pellets in Example 1, those of the invention, are administered in hard gelatine capsules and packaged in blister packs. These are kept for 6 months at 25° C., after this time the methylphenidate release profile will be reviewed.

In Table 3 the methylphenidate release profile produced at the start and after 6 months for the pellets in Example 1 are shown, expressed as % in weight of methylphenidate realeased at a certain time:

TABLE 3

| | Example 1 | Example 1 |
|---|---|---|
| Time (hours) | Time 0 | 6 months |
| 0 | 0 | 0 |
| 1 | 33.8 | 36.1 |
| 2 | 40.8 | 46.4 |
| 4 | 63.7 | 68.6 |
| 8 | 91.9 | 92.1 |
| 12 | 104.1 | 99.2 |

The similarity factor between the methylphenidate release profile after 6 months and that of the release profile at the start is more than 68%, indicating that the two release profiles are similar.

After 6 months of stability, the multi-layered pellets object of the invention are suitable for maintaining therapeutic levels in plasma for 12 hours and administering a single daily dose.

### COMPARATIVE EXAMPLE 1

Following the procedure described in Examples 1 to 4, controlled-release methylphenidate pellets are made that containing a weight ratio between the methylphenidate present in the first active layer and the ethyllcellulose of 2.19, that is not within the object of this invention. Table 4 shows the methylphenidate release profile for these pellets, expressed as % in weight of methylphenidate released at a certain time:

TABLE 4

| Time (hours) | Comparative Example 1 |
|---|---|
| 0 | 0 |
| 1 | 37.55 |
| 2 | 54.45 |
| 4 | 82.05 |
| 8 | 100.49 |
| 12 | 103.84 |

You can see that after 8 hours all of the methylphenidate present in the pellets has been released, therefore these pellets are not adequate for administering a single daily dose.

1. A controlled-release multi-layered methylphenidate pellet comprising:
an inert core,
a first active layer that is comprised of between 656 and 756 in weight of the total methylphenidate, a filmogenic substance, a plasticizer and an acid buffering system adjusted to a pH value of between 4 and 5,
a protective layer,
a layer of plastified ethylcellulose, and
a second active layer that is comprised of between 25% and 35% in weight of the total methylphenidate, a filmogenic substance, and a plasticizer,
wherein the weight ratio between the methylphenidate present in the first active layer and the ethylcellulose is between 1.40:1 and 1.90:1.

2. The controlled-release multi-layered methylphenidate pellet according to claim 1, further comprising an external coating.

3. The controlled-release multi-layered pellet according to claim 1, wherein the weight ratio between the methylphenidate present in the first active layer and the ethylcellulose is between 1.50:1 and 1.80:1.

4. The controlled-release multi-layered pellet according to claim 1, wherein the inert core is a microcrystalline cellulose inert core.

5. The controlled-release multi-layered pellet according to claim 1, wherein the inert core has a diameter of between 700 and 1000 microns.

6. The controlled-release multi-layered pellet according to claim 1, wherein the first active layer contains 70% in weight of the total methylphenidate present in the pellet.

7. The controlled-release multi-layered pellet according to claim 1, wherein in the first active layer the filmogenic substance is hydroxypropyl methylcellulose, and the plasticizer is a mixture of polyethylene glycol 400 and polyethylene glycol 6000.

8. The controlled-release multi-layered pellet according to claim 1, wherein the acid buffering system is selected from the group consisting of:
a mixture of citric acid and citrate,
a mixture of citric acid and glycine,
a mixture of glutaric acid and glycine,
a mixture of monosodium phosphate and disodium phosphate, and
a mixture of monopotassium phosphate and dipotassium phosphate.

9. The controlled-release multi-layered pellet according to claim 8, wherein the acid buffering system is a mixture of citric acid and glycine.

10. The controlled-release multi-layered pellet according to claim 1, wherein the protective layer is made of a filmogenic substance.

11. The controlled-release multi-layered pellet according to claim 10, wherein the filmogenic substance is hydroxypropyl methylcellulose.

12. The controlled-release multi-layered pellet according to claim 1, wherein the protective layer also contains a plasticizer.

13. The controlled-release multi-layered pellet according to claim 12, wherein the plasticizer is a mixture of polyethylene glycol 400 and polyethylene glycol 6000.

14. The controlled-release multi-layered pellet according to claim 1, wherein the second active layer contains 30% in weight of the total weight of methylphenidate present in the pellet.

15. The controlled-release multi-layered pellet according to claim 1, wherein in the second active layer, the filmogenic substance is hydroxypropyl methylcellulose, and the plasticizer is a mixture of polyethylene glycol 400 and polyethylene glycol 6000.

16. The controlled-release multi-layered pellet according to claim 2, wherein the pellet is coated with an external layer comprising a filmogenic substance, pigments and a plasticizer.

17. The controlled-release multi-layered pellet according to claim 1, wherein the external layer the filmogenic substance is hydroxypropyl methylcellulose, the pigment is titanium dioxide and the plasticizer is a mixture of polyethylene glycol 400 and polyethylene glycol 6000.

18. A process for preparing the controlled-release multi-layered methylphenidate pellets according to claim 1, comprising the following steps:
coating inert cores with a preliminary active layer by applying and later drying an aqueous solution that comprises between 656 and 756 in weight of the total methylphenidate, a filmogenic substance, a plasticizer and an acid buffering system adjusted to a pH value of between 4 and 5,
applying a protective layer thereto,
adding a layer of plastified ethylcellulose, so that the weight ratio between the methylphenidate present in the first active layer and the ethylcellulose is between 1.40:1 and 1.90:1, and
adding a second active layer that contains between 25% and 35% in weight of the total methylphenidate, a filmogenic substance and a plasticizer.

19. The process according to claim 18, further comprising a step of coating the pellet formed with an external coating.

20. A pharmaceutical form that contains the multi-layered pellets according to claim 1.

21. The pharmaceutical form according to claim 20, wherein the form is a capsule.

22. The pharmaceutical form according to claim 20, wherein the form contains a dose of methylphenidate hydrochloride of between 10 and 40 mg.

23. The pharmaceutical form according to claim 22, wherein the form contains a dose of 20 mg methylphenidate hydrochloride.
